(19) United States
(12) Patent Application Publication
(10) Pub. No.: US 2006/0206950 A1
(43) Pub. Date: Sep. 14, 2006

(54) In vivo imaging of e2f-fegulated bioluminescent proteins

(76) Inventor: William G. Kaelin JR., Boston, MA (US)

Correspondence Address: Ivor R. Elrifi;Mintz, Levin, Cohn, Ferris, Glovsky, and Pepeo, One Financial Center, Boston, MA, 02111, US

(21) Appl. No.: 10/533,839

(22) PCT Filed: Nov. 4, 2003
(86) PCT No.: PCT/US03/35282

§ 371 Date: Mar. 14, 2006

Related U.S. Application Data
(60) Provisional application No. 60/423,673, filed on Nov. 4, 2002.

(51) Int. Cl. A01K 67/027; C12N 5/06

(52) U.S. Cl. 800/18; 435/354

(57) ABSTRACT

The present invention relates to transgenic animals, as well as compositions and methods relating to the identification of prolifering cells. Specifically, the present invention provides transgenic mice having a nucleic acid comprising an E2F responsive promoter operably linked to a nucleic acid encoding a bioluminescent protein. Such transgenic mice are useful as models for disease and for identifying agents that modulate gene expression and are potential treatments for various disease states and disease conditions such as cancer.

### FIELD OF THE INVENTION

The invention generally relates to compositions and methods for imaging cells, e.g., cancer cells in vivo. More particularly the invention features a transgenic animal containing a polynucleotide containing a cell proliferative responsive moiety, and a reporter gene and a method of detecting proliferating cells with these transgenic animals.

### BACKGROUND OF THE INVENTION

Cell proliferation is controlled by an orderly sequence of events termed the mitotic cell cycle. Each stage of the cycle is characterized by the expression of a set of genes required to progress through that stage. Regulation of cell proliferation is central to the differentiation of specific cell types, the maintenance of tissue homeostasis and the ability of certain cell types to expand rapidly in number. The loss of cell growth control, as it occurs in the development of tumors and other pathological conditions, has profound negative effects on an organism. Tumors may develop within the interior of an organ or tissue, making early detection difficult. Methods of imaging tumor formation and growth in situ are useful to the early detection of carcinogenesis.

### SUMMARY OF THE INVENTION

The present invention generally relates to transgenic animals and methods of imaging cells (i.e., proliferating cells) using these transgenic animals. In particular, the present invention provides a transgenic mammal, e.g., non-human mammal such as a mouse or rat including as transgenes, a recombinant polynucleotide with a first nucleic acid encoding a light-generating gene product, e.g., a bioluminescent protein, such as luciferase or GFP, and a second nucleic acid constituting a cell proliferative responsive element, e.g. an E2F binding site or an E2F responsive promotor or fragment thereof. The first and second nucleic acids are operably linked. The second nucleic acid is 5′ of the first nucleic acid. Alternatively, the first nucleic acid is 5′ of the second nucleic acid. The transgenic animal is heterozygous for the transgene. Alternatively, the transgenic animal is homozygous for the transgene.

The transgene includes one, two, three, four or more E2F binding sites. The transgene also includes insulators, transcriptional terminators, and other nucleic acid sequences allowing the expression of the light generating moiety.

The cell proliferative responsive element binds polypeptides such as pRB, p107, p130, E2F1, E2F2, E2F3, E2F4, E2F5, E2F6, E2F7, G14 cyclin, and a cyclin-dependent kinase. For example, the cell proliferative responsive element is an E2F responsive promoter such as the E2F-1 promoter, dihydrofolate reductase promoter, DNA polymerase alpha promoter, c-myc promoter, cyclin E promoter, p73 promoter, or B-myb promoter. Preferably, the E2F responsive promoter is from a mammal, e.g., a human E2F-1 promoter.

The light generating gene product is a bioluminescent protein such as ferredoxin IV, a green fluorescent protein, a red fluorescent protein, a yellow fluorescent protein, a member of the luciferase family, or a member of the aequorin family.

The invention further provides cells isolated from the transgenic mammal that include the transgene.

In another aspect the invention provides a method for the production of a transgenic mammal by introducing a recombinant nucleic acid molecule including an E2F responsive promoter operably linked to a nucleic acid encoding a bioluminescent protein into a germ cell, an embryonic cell, an egg cell or a cell derived therefrom.

In yet another aspect the invention provides a method for the identification of a compound capable of modifying an activity of E2F, by contacting a transgenic mammal that contains a nucleic acid molecule including an E2F responsive promoter operably linked to a nucleic acid encoding a bioluminescent protein or a cell isolated therefrom with a test compound and measuring the light generated by the bioluminescent protein. The compound increases expression of the bioluminescent protein. Alternatively, the compound decreases expression of the bioluminescent protein. The compound affects E2F-regulated transcription by a number of mechanisms, including stimulation or inhibition of transcription factor binding, inhibition of transcription initiation, or other mechanisms.

A further aspect of the invention provides a method for detecting a proliferating cell in vivo, by administering a substrate (e.g., luciferin) to a transgenic mammal, which contains a nucleic acid molecule including an E2F responsive promoter operably linked to a nucleic acid encoding a bioluminescent protein; and measuring with a photodetector device, photon emission through opaque tissue, thereby detecting a proliferating cell.

In still a further aspect the invention provides a method for detecting a proliferating cell in vivo by contacting an isolated nucleic acid and a cell suspected of being a proliferating cell, the nucleic acid comprising an E2F responsive promoter operably linked to a nucleic acid encoding a bioluminescent protein, such that the nucleic acid enters into the cell and the bioluminescent protein is produced; and detecting the produced bioluminescent protein, thereby detecting the proliferating cell. The proliferating cell is for example a cancer cell such as brain cancer (e.g., glioma), lung cancer, liver cancer, breast cancer, Burkitts lymphoma, Hodgkin's disease, cervical cancer, ovarian cancer, or retinoblastoma. The nucleic acid includes a vector, such as plasmid vectors or viral vectors.

A further aspect of the invention relates to a non-invasive method for localizing a proliferating cell in a subject by introducing to the subject a nucleic acid including an E2F responsive promoter operably linked to a nucleic acid encoding a bioluminescent protein, such that the nucleic acid enters into the cell and the bioluminescent protein is produced; and detecting the bioluminescent protein, thereby localizing the malignant cell in the subject. The proliferating cell is a cell from a malignant cancer. This method is useful to differentiate non-cancerous tissue from cancerous tissue. For example, the method is used to differentiate malignant tumors from benign tumors.

Another aspect of the invention relates to a method for localizing cancerous tissue in a subject by introducing to the subject a nucleic acid including an E2F responsive promoter operably linked to a nucleic acid encoding a bioluminescent protein, such that the nucleic acid enters into one or more cells of the cancerous tissue, and the bioluminescent protein is produced in one or more cells; and detecting the bioluminescent protein, thereby localizing the cancerous tissue in the subject.

Another aspect of the invention relates to a method of determining the efficacy of an anti-tumor compound in a subject by introducing to the subject a nucleic acid including an E2F responsive promoter operably linked to a nucleic acid encoding a bioluminescent protein, such that the nucleic acid enters into one or more cells of the tumor tissue and the bioluminescent protein is produced in one or more cells; measuring the luminescence of the bioluminescent protein prior to administration of the anti-tumor compound; administering the anti-tumor compound to the subject; measuring the luminescence of the bioluminescent protein following administration of the anti-tumor compound; and comparing the luminescence of the bioluminescent protein prior to and following administration of the anti-tumor compound, thereby determining the efficacy of the anti-tumor compound in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic illustration of a nucleic acid construct useful in generating a transgenic mammal of the present invention.

FIG. 2 is a schematic illustration of the human E2F1 promoter region useful in the present invention.

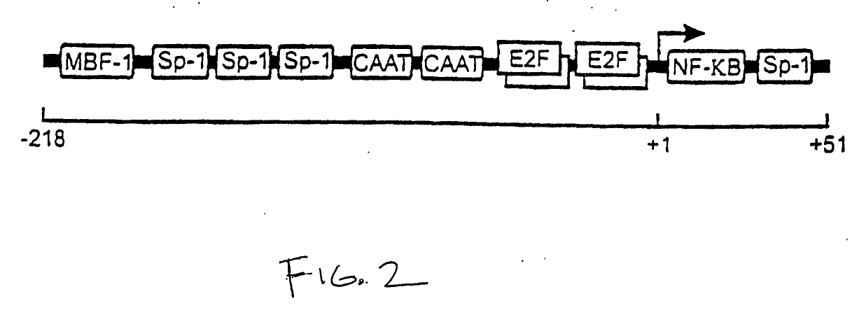

### DETAILED DESCRIPTION OF THE INVENTION

The present invention provides transgenic non-human animals which contain, as transgenes, a recombinant Polynucleotide with first nucleic acid encoding a light-generating gene product and a second nucleic acid constituting a cell proliferative responsive element, e.g., an E2F responsive promotor. Also provided are methods of imaging proliferating cells, e.g., cancer cells using the transgenic animals.

In cells, E2F activates gene expression of a number of cell cycle-dependent genes. In tumor cells, E2F is deregulated compared to proliferating non-tumor cells. Many cell-cycle regulated genes contain E2F binding sites, including many genes required for DNA replication specifically and S-phase entry/traversal generally. E2F is negatively regulated by members of the pRB (retinoblastoma protein) family. Thus free E2F is a transcriptional activator and is silenced upon binding to pRB (or its paralogs). Binding of pRB to E2F is regulated by cell-cycle dependent pRB phosphorylation. Most human tumors harbor mutations that directly or indirectly inactivate pRB. (See Sellers and Kaelin, J. Clin. One., 1997. 15: p. 3301-3312).

pRB, rather than passively sequestering E2F, converts E2F from an activator to a potent transcriptional repressor. Thus, elimination of E2F sites in various E2F-responsive promoters is known to increase, rather than decrease, transcription—presumably due to loss of pRB/E2F transcriptional repressor complexes. pRB preferentially binds to E2F1, E2F2, E2F3, and E2F4. E2F1, E2F2, and E2F3 are potent activators when not bound to pRB, whereas E2F4 is not. Furthermore, E2F1, E2F2, and E2F3 are themselves encoded by E2F-responsive promoters.

The E2F1 promoter contains 4 canonical E2F binding sites and can render the transcription of a heterologous reporter gene cell-cycle dependent in cell culture experiments (See Neuman et al., Mol. Cell. Biol., 1994. 14: p. 6607-6615; Hsiao et al., Genes Dev., 1994. 8: p. 1526-1537; Johnson et al., Genes Dev., 1994. 8: p. 1514-1525). E2F deregulation occurs during tumorigenesis. For example, an E2F-responsive viral reporter gene construct is capable of discriminating between cycling normal cells and cycling tumor cells. (See, e.g., Parr et al. Nat Med. 1997. 3(10):1145-9). This discrimination depends upon the integrity of the E2F binding sites in the E2F1 promoter.

Definitions

“Light-generating” or “luminescent” includes the property of generating light through a chemical reaction or through the absorption of radiation, including phosphorescence, fluorescence, and bioluminescence.

“Bioluminescent proteins” include any light-generating polypeptides, including fluorescent proteins such as green fluorescent protein (GFP) and luminescent proteins such as luciferase.

“Bioluminescent” molecules or moieties include luminescent substances such as proteins that utilize chemical energy to produce light.

“Fluorescent” molecules or moieties include those that are luminescent via a single electronically excited state, which is of very short duration after removal of the source of radiation. The wavelength of the emitted fluorescence light is longer than that of the exciting illumination (Stokes' Law), because part of the exciting light is converted into heat by the fluorescent molecule.

“Light” includes electromagnetic radiation having a wavelength of between about 300 nm and about 1100 nm, but can be of longer or shorter wavelength.

“Non-invasive” methods for detecting localization in a subject does not include largely invasive methods such as conventional surgery or biopsy.

“Light-generating gene product” includes any protein known to those of ordinary skill in the art to provide a readily detectable source of light when present in stable form. Non-limiting examples include light-generating proteins described in U.S. Pat. Nos. 5,683,888, 5,958,713, and 5,650,135, e.g., ferredoxin IV, green fluorescent protein, red fluorescent protein, yellow fluorescent protein, blue fluorescent protein, the luciferase family (see, e.g., Wo 03/016839), and the aequorin family. In a preferred embodiment, the light-generating polypeptide moiety is a protein such as green fluorescent protein, red fluorescent protein, yellow fluorescent protein and blue fluorescent protein. Light-generating gene products include light-generating polypeptide moieties.

“Light-generating fusion protein” includes proteins of the invention having a light-generating or luminescent portion, i.e., a light-generating polypeptide such as luciferase and a ligand binding site. In general, when a ligand of interest binds to the ligand binding site of the light-generating fusion protein, the light-generating properties of the light-generating polypeptide change, either going from undetectable (“off” or “dark”) to detectable (“on” or “light”), or vice versa.

“Ligand” includes a molecule, a small molecule, a biomolecule, a drug, a peptide, a polypeptide, a protein, a protein complex, an antibody, a nucleic acid, or a cell, which binds or otherwise interacts with a protein, antibody, receptor, or transcription factor, such as a bioluminescent protein.

“Colinear effector site” includes regions of the light-generating polypeptide moiety that, when acted on by events subsequent to ligand binding, cause the light-generating polypeptide moiety to change its present light-generating state (i.e., on or off). These regions making up the colinear effector site may do this by, e.g., conformational distortion, chemical modification, e.g., ubiquitination of a residue or residues in the colinear effector site, or by cleavage of a portion of all or part of the colinear effector site. The bioluminescent proteins of the invention may have one or more colinear effector sites.

“Localization” includes determining the particular region of the subject where an entity of interest, e.g., a tumor, resides.

“Small molecule” includes compositions that have a molecular weight of less than about 5 kD and most preferably less than about 4 kD. Small molecules is, e.g., nucleic acids, peptides, polypeptides, peptidomimetics, carbohydrates, lipids or other organic or inorganic molecules. “Spread of infection” includes the spreading and colonization by a pathogen of host sites other than the initial infection site. The term can also include, however, growth in size and/or number of the pathogen at the initial infection site.

“Ligand binding site” includes the location on the light-generating fusion protein to which a ligand binds, whereupon the light-generating polypeptide moiety is activated or inactivated as a direct or indirect consequence of ligand binding. Binding to the ligand-binding site is direct or indirect, e.g., via protein dimerization in conjunction with other proteins, as described herein.

“Targeting moiety” includes moieties such as proteins that allow the bioluminescent protein of the invention to be selectively delivered to a target organ or organs. Many targeting moieties are known, and include, for example, asialoglycoproteins (see, e.g. Wu, U.S. Pat. No. 5,166,320) and other ligands which are transported into cells via receptor-mediated endocytosis.

“Entities” include, without limitation, small molecules such as cyclic organic molecules; macromolecules such as proteins; polymers; proteins; polysaccharides; nucleic acids; particles, inert materials; organelles; microorganisms such as viruses, bacteria, yeast and fungi; cells, e.g., eukaryotic cells; embryos; prions; tumors; all types of pathogens and pathogenic substances; and particles such as beads and liposomes. In another aspect, entities are all or a portion (e.g., less than 0.01%, 0.1%, 1%, 5%, 10%, 25%, 50%, 75%, 90%, 99% or more) of the cells that constitute the mammalian subject being imaged, e.g., a diseased or injured tissue or organ, or compounds or molecules produced by those cells, or by a condition under study. Entities for which the invention has particular utility include tumors, proliferating cells, pathogens, and cellular environments comprising hypoxic tissue.

“Opaque medium” includes a medium that is “traditionally” opaque, not necessarily absolutely opaque. Accordingly, an opaque medium includes a medium that is commonly considered to be neither transparent nor translucent, and includes items such as a wood board, and tissue, e.g., skin and/or internal organs, of a mammal.

“promoter induction event” includes an event that results in the direct or indirect induction of a selected inducible promoter.

“Heterologous gene” includes a gene that has been transfected into a host organism. Typically, a heterologous gene refers to a gene that is not originally derived from the transfected or transformed cells' genomic DNA.

“Recombinant nucleic acid molecules” include nucleic acid sequences not naturally present in the cell, tissue or organism into which they are introduced.

The term “operably linked” relates to the orientation of polynucleotide elements in a functional relationship. An E2F responsive promoter is operably linked to a nucleic acid encoding a bioluminescent protein if the E2F responsive promoter regulates (e.g., promotes or represses) transcription of the bioluminescent protein. Operably linked means that the DNA sequences being linked are generally contiguous and, where necessary to join two protein coding regions, contiguous and in the same reading frame. However, since enhancers generally function when separated from the promoter by several kilobases, some nucleic acids is operably linked but not contiguous.

An “insulator” includes a nucleic acid sequence that protects a gene from inappropriate signals emanating from the surrounding environment, such as a distal enhancer.

A “transcriptional terminator” includes a DNA sequence that stops or pauses transcription.

The terms “polynucleotide” and “nucleic acid molecule” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-, double-stranded and triple helical molecules. “Oligonucleotide” refers to polynucleotides of between 5 and about 100 nucleotides of single- or double-stranded DNA. Oligonucleotides are also known as oligomers or oligos and are isolated from genes, or chemically synthesized by methods known in the art. A “primer” refers to an oligonucleotide, usually single-stranded, that provides a 3′-hydroxyl end for the initiation of enzyme-mediated nucleic acid synthesis. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine.

A “fragment” of a polynucleotide is a polynucleotide comprised of at least 9 contiguous nucleotides, preferably at least 15 contiguous nucleotides and more preferably at least 45 nucleotides, of coding or non-coding sequences.

The term “gene targeting” refers to a type of homologous recombination that occurs when a fragment of genomic DNA is introduced into a mammalian cell and that fragment locates and recombines with endogenous homologous sequences.

The term “homologous recombination” refers to the exchange of DNA fragments between two DNA molecules or chromatids at the site of homologous nucleotide sequences.

The term “homologous” as used herein denotes a characteristic of a DNA sequence having at least about 70 percent sequence identity as compared to a reference sequence, typically at least about 85 percent sequence identity, preferably at least about 95 percent sequence identity, and more preferably about 98 percent sequence identity, and most preferably about 100 percent sequence identity as compared to a reference sequence. Homology is determined using, for example, a “BLASTN” algorithm. It is understood that homologous sequences can accommodate insertions, deletions and substitutions in the nucleotide sequence. Thus, linear sequences of nucleotides are essentially identical even if some of the nucleotide residues do not precisely correspond or align. The reference sequence is a subset of a larger sequence, such as a portion of a gene or flanking sequence, or a repetitive portion of a chromosome.

The term “target gene” (alternatively referred to as “target gene sequence” or “target DNA sequence” or “target sequence”) refers to any nucleic acid molecule, polynucleotide, or gene to be modified by homologous recombination. The target sequence includes an intact gene, an exon or intron, a regulatory sequence or any region between genes. The target gene may comprise a portion of a particular gene or genetic locus in the individual's genomic DNA.

The term “transgenic cell” refers to a cell containing within its genome an E2F responsive promoter operably linked to a nucleic acid encoding a bioluminescent protein introduced by the method of gene targeting.

The term “proliferating cell” includes any cell undergoing cell division.

The term “cancer-associated cell” refers to a cell that can be isolated from a cell population containing one or more tumor cells, including cells that can be isolated from a solid, soft, hematologic or other tumor.

The term “transgenic animal” refers to an animal that contains within its genome a specific gene that has been disrupted or otherwise modified or mutated by the method of gene targeting. “Transgenic animal” includes both the heterozygous animal (i.e., one allele bearing the heterologous nucleic acid and one wild-type allele) and the homozygous animal (i.e., two alleles bearing the heterologous nucleic acid).

As used herein, the terms “selectable marker” and “positive selection marker” refer to a gene encoding a product that enables only the cells that carry the gene to survive and/or grow under certain conditions. For example, plant and animal cells that express the introduced neomycin resistance (Neo (r)) gene are resistant to the compound G418. Cells that do not carry the Neo (r) gene marker are killed by G418. Other positive selection markers are known to or are within the purview of those of ordinary skill in the art.

A “host cell” includes an individual cell or cell culture that is or has been a recipient for vector(s) or for incorporation of nucleic acid molecules and/or proteins. Host cells include progeny of a single host cell, and the progeny may not necessarily be completely identical (in morphology or in total DNA complement) to the original parent due to natural, accidental, or deliberate mutation. A host cell includes cells transfected with the constructs of the present invention.

The term “modulates” as used herein refers to the decrease, inhibition, reduction, increase, or enhancement of a gene function, expression, activity, or alternatively, a phenotype associated with an E2F responsive promoter operably linked to a nucleic acid encoding a bioluminescent protein.

The invention relates in part to methods and compositions relating to detecting, localizing and quantifying cell cycle or cell proliferation-related activities in vivo using transgenic animals expressing bioluminescent proteins controlled by an E2F responsive promoter. Light emission is detected by known methods, such as detection with suitable instrumentation (such as a CCD camera) in vivo or in vitro, such as in a living cell or intact organism, a cell culture system, a tissue section, or an array.

Transgenic Mammals.

The transgenic animals of the present invention are produced with a transgene which includes a recombinant polynucleotide with a first nucleic acid encoding a reporter gene product (a light-generating gene) operably linked to a second nucleic acid constituting a cell proliferative responsive element. Alternatively, the transgene contains two, three, four or five or more cell proliferative responsive elements. The transgene is integrated into the genome of the transgenic animal. For example, the transgene is stably integrated into the animal's genome. Optionally, the transgene is transiently integrated into the animal's genome. Alternatively, the transgene is not integrated into the genome of the transgenic animal (i.e., remains in an episomal state). The transgene is introduced into the animal, for example, by pronuclear injection or targeted insertion.

By “cell proliferative responsive element” is meant that the element is responsive to cell proliferative signals. For example, the cell proliferative responsive element is an E2F responsive promoter. An E2F responsive promoters includes any nucleic acid sequence that binds to a member of the E2F family, including E2F1-E2F6 and other E2F family members, as well as other polypeptides including, e.g., pRB, p107, p130, G1 cyclin, cyclin-dependent kinases, and E1A. E2F responsive promoters include the E2F-1 promoter, the p73 promoter, the dihydrofolate reductase (DHFR) promoter, the DNA polymerase alpha promoter, the thymidine kinase promoter, the thymidylate synthase promoter, the cyclin A promoter, the Cdc 2 promoter, the retinoblastoma gene (Rb) product promoter, the c-myc promoter, or the b-myb promoter. Preferably, the E2F-responsive promoter is the human E2F1 promoter. An exemplary cell proliferative responsive element includes the human E2F1 promoter of SEQ ID NO:1. ggtaccatccggacaaagcctgcgcgcgccccgccccgccattggccgtaccgccccgcgccgccgccccatctcgcccctcgccgccgggtccggcgcgttaaagccaataggaaccgccgccgttgttcccgtcacggccggggcagccaattgtggcggcgctcggcggctcgtggctctttcgcggcaaaaaggatttggcgcgtaaaagtggccgggactttgcaggcagcggcggccgggggcggagcgggatcgagccctcg (SEQ ID NO: 1) (Corresponding to nucleotides −218 to +51 of the human E2F1 gene, GenBank Accession No. AF516106. (See, e.g., Parr et al. Nature Medicine, 1997. 3:1145-1149) relative to the translation start site (See Neuman et al Mol Cell Biol, 1995. 15(8): p. 4660). Additional E2F responsive promoters include the human DHFR promoter (NM-000791); the mouse DHFR promoter (NM_{—}010049); the human thymidine kinase promoter (NM_{—}003258 and M13643); the human DNA polymerase alpha promoter (X66868); the human b-myb promoter (X82032); and the human thymidylate synthase promoter (AF134214 and AD134215).

Alternatively, the cell proliferative responsive element is a synthetic E2F-responsive promoter containing one or more canonical E2F binding sites (e.g., two, three, four, five, six, seven, eight or more canonical E2F binding sites). For example, a cell proliferative responsive element includes the consensus sequence TTT(G/C)(G/C)CG(G/C) (SEQ ID NO:2). Optionally, a cell proliferative responsive element includes the consensus sequence TTT(G/C)(G/C)CG(G/C)NTTT(G/C)(G/C)CG(G/C) (SEQ ID NO:3); TTT(G/C)(G/C)CG(G/C)NTTT(G/C)(G/C)CG(G/C)NTTT(G/C)(G/C)CG(G/C) (SEQ ID NO:4); or TTT(G/C)(G/C)CG(G/C)NTTT(G/C)(G/C)CG(G/C) NTTT(G/C)(G/C)CG(G/C)NTTT(G/C)(G/C)CG(G/C) (SEQ ID NO: 5), where N is any one or more nucleotides, or is not a nuceotide.

Endogenous E2F-responsive promoters can also be employed to express a DNA encoding a bioluminescent protein. For example, a nucleic acid encoding luciferase is introduced into a gene having an E2F-responsive promoter, such that the luciferase gene is expressed when the E2F promoter is activated.

In general, any light-generating moiety that gives off light can be used. The selection of a light generating protein should be done so as to produce light capable of penetrating animal tissue such that it is detected externally in a non-invasive manner. The ability of light to pass through a medium such as animal tissue (composed mostly of water) is determined primarily by the light's intensity and wavelength. The more intense the light produced in a unit volume, the easier the light will be to detect. The intensity of light produced in a unit volume depends on the spectral characteristics of individual bioluminescent proteins, and on the concentration of those proteins in the unit volume. A second factor governing detectability through a layer of tissue is the wavelength of the emitted light.

Accordingly, light generating proteins that emit light in the range of yellow to red (550-1100 nm) are typically preferable to those which emit at shorter wavelengths. However, excellent results are achieved in practicing the present invention with bioluminescent proteins that emit in the range of 486 nm, despite the fact that this is not an optimal emission wavelength.

The light-generating moieties are bioluminescent proteins, such as luciferase (e.g., firefly [Photinus pyralis] luciferase or renilla luciferase), obelin and aequorin. Preferred light-generating moieties include firefly luciferase, which has been used for the quantitative determination of specific substances in biology and medicine for many years. In other embodiments, the light-generating moiety is a fluorescent protein, e.g., green fluorescent protein (GFP).

The transgene as described herein can optionally contain one or more additional nucleic acids sequences such as homologous flanking sequences to facilitate homologous recombination, a selection marker e.g., negative or positive), an insulator sequence or a transcriptional terminator sequence. The transgene optionally includes tissue specific promoter sequences (i.e., promoters that cause expression of the transgene in a specific type of cells, tissues, or organs).

The size of the homologous flanking sequences are not critical and can range from as few as about 15-20 base pairs to as many as 100 kb, such as about 1 kb. In some embodiments each fragment is greater than about 1 kb in length, between about 1 and about 10 kb, or between about 1 and about 5 kb. One of skill in the art will recognize that although larger fragments may increase the number of homologous recombination events in ES cells, larger fragments will also be more difficult to clone.

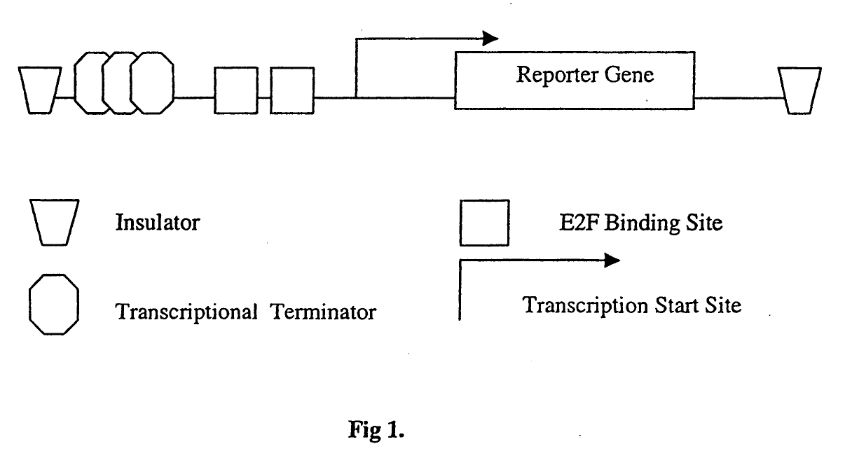

By an “insulator sequence” is meant that the sequence protects an expressed gene from surrounding influences by either blocking the action of a distal enhancer on a promoter or preventing the advance of nearby condensed chromatin that might otherwise silence expression. As shown in FIG. 1, insulator elements are for example situated 5′ of the E2F responsive promoter and 3′ of the nucleic acid encoding the bioluminescent protein. Insulators useful in the present invention are isolated from humans (e.g;, 5′HSS, DMD/ICR, apoB, and DMI); mice (BEAD-1, HS2-6, and DMD/ICR); chicken (Lys 5′A, HS4, and 3′HS); xenopus (RO); fruit fly (scs, scs′, gypsy, Fab-7, Fab-8, fa^{swb}, and eve promoter); and yeast (HMR tRNA^{Thr}, Cha1 UAS, and STAR).

By “transcriptional terminator” is meant that the sequence acts as a signal for termination of transcription. A transcriptional terminator includes Rho-independant terminators (a stem-loop structure in the transcribed RNA followed by a run of U residues) or Rho-dependant terminators (unstructured region of RNA that, when untranslated, is recognized by Rho factor). Exemplary transcriptional terminators include the yeast Reb1 terminator, the E. coli glmS transcriptional terminator; the aspA terminator, the SV40 terminator, and the THP terminator (See, e.g., Krebber et al., 1996. Gene 178:71-74).

By “selection marker” is meant any gene encoding a gene product that allows for selection of the cell carrying the transgene. Such selection systems are well known in the art. Examples of selectable markers include, but are not limited to, known genes encoding resistance to antibiotics such as the aminoglycoside antibiotics (including, e.g, neomycin, hygromycin, kanamycin, bleomycin, G418). A suitable marker for use in aminoglycoside based selection is the neomycin phosphotransferase (neo) gene (see, e.g., Potrykus et al., Mol. Gen. Genet. 199:183-188 (1985). Other selectable markers include the dihydrofolate reductase (DHFR) gene, which confers resistance to methotrexate (Thillet et al., J. Biol. Chem. 263:12500-12508 (1988), or selection systems based on resistance to puromycin, blasticidin, or zeocin resistance. Other selectable markers include GFP and LacZ. In this case, the GFP/LacZ can be used both for selection by fluorescence-activated cell sorting, as well as for detection. Optionally, the selection marker is a combination of two or selection markers. For example, a positive selection marker and a negative selection marker are used (such as neomycin (or its analog G418) used as a positive selection marker and diphtheria toxin (e.g., the diphtheria toxin A-fragment) used as a negative selection marker). See, e.g., U.S. Pat. No. 5,464,764 to Capecchi, which is incorporated herein by reference in its entirety.

The transgenic animals of the present invention are generated using standard methods known in the art and as set forth in U.S. Pat. Nos. 5,614,396 5,487,992, 5,464,764, 5,387,742, 5,347,075, 5,298,422, 5,288,846, 5,221,778, 5,175,384, 5,175,383, 4,873,191, and 4,736,866.

Generation of Recombinant Nucleic Acid Molecules

The recombinant nucleic acid molecule (transgene) is produced using standard methods known in the art. (see, e.g., Sambrook, et al., 1989, Molecular Cloning: A Laboratory Manual, Second Edition, Cold Spring Harbor Laboratory Press, Cold Spring Harbor, N.Y.; E. N. Glover (eds.), 1985, DNA Cloning: A Practical Approach, Volumes I and II; M. J. Gait (ed.), 1984, Oligonucleotide Synthesis; B. D. Hames & S. J. Higgins (eds.), 1985, Nucleic Acid Hybridization; B. D. Hames & S. J. Higgins (eds.), 1984, Transcription and Translation; R. I. Freshney (ed.), 1986, Animal Cell Culture; Immobilized Cells and Enzymes, IRL Press, 1986; B. Perbal, 1984, A Practical Guide To Molecular Cloning; F. M. Ausubel et al., 1994, Current Protocols in Molecular Biology, John Wiley & Sons, Inc.). For example, the recombinant nucleic acid molecule is prepared in accordance with conventional ways, where the sequences is synthesized, isolated from natural sources, manipulated, cloned, ligated, subjected to in vitro mutagenesis, primer repair, or the like. At various stages, the joined sequences are cloned, and analyzed by restriction analysis, sequencing, or the like.

The targeting DNA is constructed using techniques well known in the art. For example, the targeting DNA is produced by chemical synthesis of oligonucleotides, polymerase chain-reaction amplification of a sequence (or ligase chain reaction amplification), purification of prokaryotic or target cloning vectors harboring a sequence of interest (e.g., a cloned cDNA or genomic DNA, synthetic DNA or from any of the aforementioned combination) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Moreover, the length of homology is selected using known methods in the art. For example, selection is based on the sequence composition and complexity of the predetermined endogenous target DNA sequence(s).

Generation of Cells and Confirmation of Homologous Recombination Events

Once an appropriate recombinant nucleic acid molecule has been prepared, the recombinant nucleic acid molecule is introduced into an appropriate host cell using any method known in the art. Various techniques are employed in the present invention, including, for example: pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g., polybrene, polyomithine, etc., or the like (see, e.g., U.S. Pat. No. 4,873,191; Van der Putten, et al., 1985, Proc. Natl. Acad. Sci., USA 82:6148-6152; Thompson, et al., 1989, Cell 56:313-321; Lo, 1983, Mol Cell. Biol. 3:1803-1814; Lavitrano, et al., 1989, Cell, 57:717-723). Various techniques for transforming mammalian cells are known in the art. (see, e.g., Gordon, 1989, Intl. Rev. Cytol., 115:171-229; Keown et al., 1989, Methods in Enzymology; Keown et al., 1990, Methods and Enzymology, Vol. 185, pp. 527-537; Mansour et al., 1988, Nature, 336:348-352).

In a preferred aspect of the present invention, the recombinant nucleic acid molecule is introduced into host cells by electroporation. In this process, electrical impulses of high field strength reversibly permeabilize biomembranes allowing the introduction of the construct. The pores created during electroporation permit the uptake of macromolecules such as DNA. (see, e.g., Potter, H., et al., 1984, Proc. Nat'l. Acad. Sci. U.S.A. 81:7161-7165).

Any cell type capable of homologous recombination is used in the practice of the present invention. Examples of such target cells include cells derived from vertebrates including mammals such as humans, bovine species, ovine species, murine species, simian species, and ether eukaryotic organisms such as filamentous fungi, and higher multicellular organisms such as plants.

Preferred cell types include embryonic stem (ES) cells, which are typically obtained from pre-implantation embryos cultured in vitro. (see, e.g., Evans, M. J., et al., 1981, Nature 292:154-156; Bradley, M. O., et al., 1984, Nature 309:255-258; Gossler et al., 1986, Proc. Natl. Acad. Sci. USA 83:9065-9069; and Robertson, et al., 1986, Nature 322:445-448). The ES cells are cultured and prepared for introduction of the recombinant nucleic acid molecule using methods well known to the skilled artisan. (see, e.g., Robertson, E. J. ed. “Teratocarcinomas and Embryonic Stem Cells, a Practical Approach”, IRL Press, Washington D.C., 1987; Bradley et al., 1986, Current Topics in Devel. Biol. 20:357-371; by Hogan et al., in “Manipulating the Mouse Embryo”: A Laboratory Manual, Cold Spring Harbor Laboratory Press, Cold Spring Harbor N.Y., 1986; Thomas et al., 1987, Cell 51:503; Koller et al., 1991, Proc. Natl. Acad. Sci. USA., 88:10730; Dorin et al., 1992, Transgenic Res. 1:101; and Veis et al., 1993, Cell 75:229). The ES cells that will be inserted with the recombinant nucleic acid molecule are derived from an embryo or blastocyst of the same species as the developing embryo into which they are to be introduced. ES cells are typically selected for their ability to integrate into the inner cell mass and contribute to the germ line of an individual when introduced into the mammal in an embryo at the blastocyst stage of development. Thus, any ES cell line having this capability is suitable for use in the practice of the present invention.

After the recombinant nucleic acid molecule has been introduced into cells, the cells in which successful gene targeting has occurred are identified. Insertion of the recombinant nucleic acid molecule into the targeted gene is typically detected by identifying cells for expression of the marker gene. In a preferred embodiment, the cells transformed with the recombinant nucleic acid molecule of the present invention are subjected to treatment with an appropriate agent that selects against cells not expressing the selectable marker. Only those cells expressing the selectable marker gene survive and/or grow under certain conditions. For example, cells that express the introduced neomycin resistance gene are resistant to the compound G418, while cells that do not express the neo gene marker are killed by G418. If the recombinant nucleic acid molecule also comprises a screening marker such as GFP, homologous recombination is identified through screening cell colonies under a fluorescent light. Cells that have undergone homologous recombination will have deleted the GFP gene and will not fluoresce, or will fluoresce in a decreased amount due to basal expression of the transgene. As used herein, “basal expression” of the transgene includes the minimal amount of E2F responsive promoter-controled expression of the bioluminescent protein in a non-proliferating (e.g., a non-cancerous) cell.

If a regulated positive selection method is used in identifying homologous recombination events, the recombinant nucleic acid molecule is designed so that the expression of the selectable marker gene is regulated in a manner such that expression is inhibited following random integration but is permitted (derepressed) following homologous recombination. More particularly, the transfected cells are screened for expression of the neo gene, which requires that (1) the cell was successfully electroporated, and (2) lac repressor inhibition of neo transcription was relieved by homologous recombination. This method allows for the identification of transfected cells and homologous recombinants to occur in one step with the addition of a single drug.

Alternatively, a positive-negative selection technique is used to select homologous recombinants. This technique involves a process in which a first drug is added to the cell population, for example, a neomycin-like drug to select for growth of transfected cells, i.e. positive selection. A second drug such as FIAU is subsequently added to kill cells that express the negative selection marker, i.e. negative selection. Cells that contain and express the negative selection marker are killed by a selecting agent, whereas cells that do not contain and express the negative selection marker survive. For example, cells with non-homologous insertion of the construct express HSV thymidine kinase and therefore are sensitive to the herpes drugs such as gancyclovir (GANC) or FIAU (1-(2-deoxy 2-fluoro-B-D-arabinofluranosyl)-5-iodouracil). (see, e.g., Mansour et al., Nature 336:348-352: (1988); Capecchi, Science 244:1288-1292, (1989); Capecchi, Trends in Genet. 5:70-76 (1989)).

Successful recombination is identified by analyzing the DNA of the selected cells to confirm homologous recombination. Various techniques known in the art, such as PCR and/or Southern analysis are used to confirm homologous recombination events.

Homologous recombination may also be used to introduce the gene constructs of the invention in stem cells, and other cell types, which are not totipotent embryonic stem cells. By way of example, stem cells are myeloid, lymphoid, or neural progenitor and precursor cells. Stem cells are derived from any vertebrate species, such as mouse, rat, dog, cat, pig, rabbit, human, non-human primates and the like.

Production of Transgenic Animals

Selected cells are then injected into a blastocyst (or other stage of development suitable for the purposes of creating a viable animal, such as, for example, a morula) of an animal (e.g., a mouse) to form chimeras (see e.g., Bradley, A. in Teratocarcinomas and Embryonic Stem Cells: A Practical Approach, E. J. Robertson, ed., IRL, Oxford, pp. 113-152 (1987)). Alternatively, selected ES cells are allowed to aggregate with dissociated mouse embryo cells to form the aggregation chimera. A chimeric embryo can then be implanted into a suitable pseudopregnant female foster animal and the embryo brought to term. Chimeric progeny harboring the homologously recombined DNA in their germ cells are used to breed animals in which all cells of the animal contain the homologously recombined DNA. In one embodiment, chimeric progeny mice are used to generate a mouse with a heterozygous recombinant nucleic acid molecule, which includes an E2F responsive promoter operably linked to a nucleic acid encoding a bioluminescent protein. Heterozygous transgenic mice can then be mated. It is well known in the art that typically ¼ of the offspring of such matings will have be homozygous for the recombinant nucleic acid molecule.

The heterozygous and homozygous transgenic mice can then be compared to normal, wild type mice to determine whether introduction of an E2F responsive promoter operably linked to a nucleic acid encoding a bioluminescent protein causes phenotypic changes, especially pathological changes. For example, heterozygous and homozygous mice are evaluated for phenotypic changes by physical examination, necropsy, histology, clinical chemistry, complete blood count, body weight, organ weights, and cytological evaluation of bone marrow. Phenotypic changes may also comprise behavioral modifications or abnormalities.

Alternatively, pronuclear injection is used to produce a transgenic animal. See, e.g., U.S. Pat. No. 4,873,191, which is incorporated herein by reference in its entirety. Pronuclear injection is when the transgene is integrated in a random fashion into the host's genome by injecting it into the pronucleus of a fertilized ovum. The DNA is capable of integration anywhere in the genome, and multiple copies often integrate in a head-to-tail fashion. The transgene contains one or more insulators. There is no need for homology between the injected transgene and the host genome. Typically, fertilized ova for the microinjection of the transgene are obtained from gonadotrophin-superovulated female mice. Hybrid strains (e.g., CB6) or inbred strains of mice are used. The transgene is introduced into the pronucleus of a fertilized ovum by microinjection. The embryos, once injected, are reimplanted into the oviduct of pseudopregnant female recipients transgene integration is assessed by tail tissue analysis or other means known in the art.

The present invention further provides transgenic animals generated by pronuclear injection of a retroviral vector containing the human E2F1 promoter and a cDNA sequence encoding firefly luciferase in to the perivitelline space of an unfertilized mammalian egg. See, e.g., U.S. Pat. No. 6,548,740, which is incorporated herein by reference in its entirety.

Lentiviral vectors are useful in to generate transgenic mice. Generally, engineered lentiviral particles are microinjected directly into the perivitelline space of mouse embryos 0.5 days after fertilization. The viral particles are comprised of a self-inactivating viral vector containing an E2F responsive promoter operably linked to a nucleic acid encoding a bioluminescent protein, as well as a marker gene in some cases. The particles also contain reverse transcriptase to catalyze the incorporation of the vector sequence into the genome, and the viral particle has a glycoprotein coat that mediates its adherence to the embryo. The viral vector incorporates into the genome of the one-celled embryo, carrying the transgene of interest with it. Two-cell stage embryos are implanted into the oviduct of pseudopregnant female mice, and resulting transgenic mice are genotyped.

Other vectors useful to generate transgenic mice include adenoviral vectors, yeast artifical chromosome (YAC) vectors, bacterial artifical chromosome (BAC) vectors, and papillomavirus-based vectors.

The transgenic animals of the present invention can be generated using gene targeting in embryonic stem cells See, e.g., U.S. Pat. No. 5,614,396.

Conditional Transgenic Animals

The present invention further contemplates conditional transgenic animals, such as those produced using recombination methods. Bacteriophage P1 Cre recombinase and flp recombinase from yeast plasmids are two non-limiting examples of site-specific DNA recombinase enzymes that cleave DNA at specific target sites (lox P sites for cre recombinase and frt sites for flp recombinase) and catalyze a ligation of this DNA to a second cleaved site. A large number of suitable alternative site-specific recombinases have been described, and their genes are used in accordance with the method of the present invention. Such recombinases include the Int recombinase of bacteriophage λ (with or without Xis) (Weisberg, R. et al., in Lambda II, (Hendrix, R., et al., Eds.), Cold Spring Harbor Press, Cold Spring Harbor, N.Y., pp. 211-50 (1983), herein incorporated by reference); TpnI and the β-lactamase transposons (Mercier, et al., J. Bacteriol., 172:3745-57 (1990)); the Tn3 resolvase (Flanagan & Fennewald J. Molec. Biol, 206:295-304 (1989); Stark, et al., Cell, 58:779-90 (1989)); the yeast recombinases (Matsuzaki, et al., J. Bacteriol, 172:610-18 (1990)); the B. subtilis SpoIVC recombinase (Sato, et al., J. Bacteriol. 172:1092-98 (1990)); the Flp recombinase (Schwartz & Sadowski, J. Molec. Biol, 205:647-658 (1989); Parsons, et al., J. Biol. Chem., 265:4527-33 (1990); Golic & Lindquist, Cell, 59:499-509 (1989); Amin, et al., J. Molec. Biol., 214:55-72 (1990)); the Hin recombinase (Glasgow, et al., J. Biol. Chem., 264:10072-82 (1989)); immunoglobulin recombinases (Malynn, et al., Cell, 54:453-460 (1988)); and the Cin recombinase (Haffter & Bickle, EMBO J., 7:3991-3996 (1988); Hubner, et al., J. Molec. Biol, 205:493-500 (1989)), all herein incorporated by reference. Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J. 8:2425-33 (1989)); Hunger-Bertling, et al., (Mol. Cell. Biochem., 92:107-16 (1990)); and Cregg & Madden (Mol. Gen. Genet., 219:320-23 (1989)), all herein incorporated by reference.

Cre has been purified to homogeneity, and its reaction with the loxp site has been extensively characterized (Abremski & Hess J. Mol. Biol. 259:1509-14 (1984), herein incorporated by reference). Cre protein has a molecular weight of 35,000 and is obtained commercially from New England Nuclear/Du Pont. The cre gene (which encodes the Cre protein) has been cloned and expressed (Abremski, et al., Cell 32:1301-11 (1983), herein incorporated by reference). The Cre protein mediates recombination between two loxP sequences (Sternberg, et al., Cold Spring Harbor Symp. Quant. Biol. 45:297-309 (1981)), which are present on the same or different DNA molecule. Because the internal spacer sequence of the loxP site is asymmetrical, two loxP sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al., Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

In one embodiment of the present invention, the recombinant nucleic acid molecule is prepared by cloning a cDNA including an E2F responsive promoter operably linked to a nucleic acid encoding a bioluminescent protein into the pBIG T vector, thereby placing it downstream of a floxed neo tPA (triple polyadenylation site) cassette. A restriction fragment spanning the Neo cassette and the luciferase transcriptional unit is subcloned into pROSA26PA, which contains flanking sequences from the ROSA 26 locus, to facilitate homologous recombination. The completed construct is typically a circular plasmid.

In one embodiment, purified recombinase enzyme is provided to the cell by direct microinjection. In another embodiment, recombinase is expressed from a co-transfected construct or vector in which the recombinase gene is operably linked to a functional promoter. An additional aspect of this embodiment is the use of tissue-specific or inducible recombinase constructs that allow the choice of when and where recombination occurs. One method for practicing the inducible forms of recombinase-mediated recombination involves the use of vectors that use inducible or tissue-specific promoters or other gene regulatory elements to express the desired recombinase activity. The inducible expression elements are preferably operatively positioned to allow the inducible control or activation of expression of the desired recombinase activity. Examples of such inducible promoters or other gene regulatory elements include, but are not limited to, tetracycline, metallothionine, ecdysone, and other steroid-responsive promoters, rapamycin responsive promoters, and the like (No, et al., Proc. Natl. Acad. Sci. USA, 93:3346-51 (1996); Furth, et al., Proc. Natl. Acad. Sci. USA, 91:9302-6 (1994)). Additional control elements that are used include promoters requiring specific transcription factors such as viral, promoters. Vectors incorporating such promoters would only express recombinase activity in cells that express the necessary transcription factors.

One skilled in the art will recognize that the recombinant nucleic acid molecules of the invention can also be introduced in animal cells in vivo by viral or non-viral DNA-based vector systems.

Diagnosis of Cell Proliferation-Associated Disorders in Human Patients.

The present invention also provides for the introduction of an E2F-responsive transgene into humans for diagnosis of cell proliferation-associated disorders, such as cancer. Cell proliferative disorders are diagnosed by introducing to a human a vector (e.g., an adenoviral vector) including an E2F-responsive promoter operably linked to a nucleic acid encoding a bioluminescent protein (e.g., luciferase). Detection of the bioluminescent protein above normal control (i.e., background) levels indicates the presence of a cell proliferation disorder in the human. Normal control levels are determined by measuring the levels of the bioluminescent protein in cells known to be not cancerous or not proliferating. Detection of the expressed bioluminescent protein is imaged using fiber-optic or endoscopic procedures known in the art (e.g., an arthroscope for imaging of a joint such as a knee; a hysteroscope for imaging of the uterus and cervix; a cystoscope for imaging of the bladder; a tracheoscope for imaging of the trachea; or a urethroscope for imaging the uretra).

Implantation of Cells Containing an E2F-Responsive Transgene into Non-Human Mammals.

The present invention also provides for the introduction of one or more cells isolated from a mammal (the donor mammal) containing an E2F-responsive transgene into an acceptable non-human mammal (the target mammal), such as a mouse (e.g., an athymic nude mouse). Generation of cells containing an E2F-responsive transgene is discussed above. The donor mammal is the same species as the target mammal. Alternatively, the donor mammal is a different species from the target mammal. For example, one or more cells isolated from a human (such as cells isolated from a tumor or suspected tumor) are contacted with an E2F-responsive transgene, which enters the cell or population of cells (e.g. by infection or transfection means). The transgene-containing cell or cell population is implanted into an athymic nude mouse. Implantation into a tissue or organ of the mammal is performed by standard surgical methods known in the art, such as by subdermally, peritoneally, subcutaneously, intravenously, or directly into an organ or tissue of interest

Imaging of Bioluminescent Proteins.

Bioluminescent proteins are imaged by methods known in the art. Since the imaging, or measuring of photon emission from the subject, may last up to tens of minutes, the subject is desirably immobilized during the imaging process. Imaging of the bioluminescent protein involves the use of, e.g., a photodetector capable of detecting extremely low levels of light—typically single photon events—and integrating photon emission until an image is constructed. Examples of such sensitive photodetectors include devices that intensify the single photon events before the events are detected by a camera, and cameras (cooled, for example, with liquid nitrogen) that are capable of detecting single photons over the background noise inherent in a detection system.

Once a photon emission image is generated, it is typically superimposed on a reflected light image of the subject to provide a frame of reference for the source of the emitted photons (i.e., localize the bioluminescent protein with respect to the subject). Such an image is then analyzed to determine the location and/or amount of a target in the mammal.

Bioluminescent proteins that have localized to their intended sites in a mammal are imaged in a number of ways. A reasonable estimate of the time to image the localization is made by one skilled in the art. For example, a mammal containing an E2F-responsive promoter operably linked to a nucleic acid encoding a luciferase is imaged about 10 to 15 minutes after addition of luciferin or other luciferase substrate to the mammal. Alternatively, the bioluminescent protein is imaged 5 minutes, 20 minutes, 30 minutes, 1 hour, 1 day, or greater than one day after addition of luciferin or other luciferase substrate. Furthermore, the state of localization as a function of time is followed by imaging the bioluminescent proteins according to the methods of the invention.

The “photodetector device” used should have a high enough sensitivity to enable the imaging of faint light from within a mammal in a reasonable amount of time, and to use the signal from such a device to construct an image.

In cases where it is possible to use bioluminescent proteins which are extremely bright, and/or to detect bioluminescent proteins localized near the surface of the mammal being imaged, a pair of “night-vision” goggles or a standard high-sensitivity video camera, such as a Silicon Intensified Tube (SIT) camera (e.g., from Hammamatsu Photonic Systems, Bridgewater, N.J.), are used. More typically, however, a more sensitive method of light detection is required.

In extremely low light levels the photon flux per unit area becomes so low that the scene being imaged no longer appears continuous. Instead, it is represented by individual photons that are both temporally and spatially distinct form one another. Viewed on a monitor, such an image appears as scintillating points of light, each representing a single detected photon. By accumulating these detected photons in a digital image processor over time, an image is acquired and constructed. In contrast to conventional cameras where the signal at each image point is assigned an intensity value, in photon counting imaging the amplitude of the signal carries no significance. The objective is to simply detect the presence of a signal (photon) and to count the occurrence of the signal with respect to its position over time.

At least two types of photodetector devices, described below, can detect individual photons and generate a signal that is analyzed by an image processor. Reduced-Noise Photodetection Devices achieve sensitivity by reducing the background noise in the photon detector, as opposed to amplifying the photon signal. Noise is reduced primarily by cooling the detector array. The devices include charge coupled device (CCD) cameras referred to as “backthinned”, cooled CCD cameras. In the more sensitive instruments, the cooling is achieved using, for example, liquid nitrogen, which brings the temperature of the CCD array to approximately −120° C. “Backthinned” refers to an ultra-thin backplate that reduces the path length that a photon follows to be detected, thereby increasing the quantum efficiency. A particularly sensitive backthinned cryogenic CCD camera is the “TECH 512”, a series 200 camera available from Photometrics, Ltd. (Tucson, Ariz.).

“Photon amplification devices” amplify photons before they hit the detection screen. This class includes CCD cameras with intensifiers, such as microchannel intensifiers. A microchannel intensifier typically contains a metal array of channels perpendicular to and co-extensive with the detection screen of the camera. The microchannel array is placed between the mammal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera.

Even greater sensitivity is achieved by placing intensifying microchannel arrays in series, so that electrons generated in the first stage in turn result in an amplified signal of electrons at the second stage. Increases in sensitivity, however, are achieved at the expense of spatial resolution, which decreases with each additional stage of amplification. An exemplary microchannel intensifier-based single-photon detection device is the C2400 series, available from Hamamatsu.

Image Processors process signals generated by photodetector devices which count photons in order to construct an image which is, for example, displayed on a monitor or printed on a video printer. Such image processors are typically sold as part of systems which include the sensitive photon-counting cameras described above, and accordingly, are available from the same sources. The image processors are usually connected to a personal computer, such as an IBM-compatible PC or an Apple Macintosh (Apple Computer, Cupertino, Calif.), which may or may not be included as part of a purchased imaging system. Once the images are in the form of digital files, they are manipulated by a variety of image processing programs (such as “ADOBE PHOTOSHOP”, Adobe Systems, Adobe Systems, Mt. View, Calif.) and printed.

The “detection field of the device” is defined as the area from which consistent measurements of photon emission is obtained. In the case of a camera using an optical lens, the detection field is simply the field of view accorded to the camera by the lens. Similarly, if the photodetector device is a pair of “night vision” goggles, the detection field is the field of view of the goggles.

Alternatively, the detection field is a surface defined by the ends of fiber-optic cables arranged in a tightly-packed array. The array is constructed to maximize the area covered by the ends of the cables, as opposed to void space between cables, and placed in close proximity to the mammal. For instance, a clear material such as plexiglass is placed adjacent the mammal, and the array fastened adjacent the clear material, opposite from the mammal.

The fiber-optic cable ends opposite the array are connected directly to the detection or intensifying device, such as the input end of a microchannel intensifier, eliminating the need for a lens. An advantage of this method is that scattering and/or loss of photons is reduced by eliminating a large part of the air space between the mammal and the detector, and/or by eliminating the lens. Even a high-transmission lens transmits only a fraction of the light reaching the front lens element.

With higher-intensity bioluminescent proteins, photodiode arrays are used to measure photon emission. A photodiode array is incorporated into a relatively flexible sheet, enabling the practitioner to partially “wrap” the array around the mammal. This approach also minimizes photon loss, and in addition, provides a means of obtaining three-dimensional images of the bioluminescence. Other approaches are used to generate three-dimensional images, including multiple detectors placed around the mammal or a scanning detector or detectors.

It will be understood that the entire mammal need not necessarily be in the detection field of the photodetection device. For example, if one is measuring a bioluminescent protein known to be localized in a particular region of the mammal, only light from that region, and a sufficient surrounding “dark” zone, need be measured to obtain the desired information.

Immobilizing the Mammal.

In those cases where it is desired to generate a two-dimensional or three-dimensional image of the mammal, the mammal (e.g., the transgenic mammal) is immobilized in the detection field of the photodetection devices during the period that photon emission is being measured. If the signal is sufficiently bright that an image is constructed from photon emission measured in less than about 20 milliseconds, and the mammal is not particularly agitated, no special immobilization precautions is required, except to insure that the mammal is in the field of the detection device at the start of the measuring period.

If, on the other hand, the photon emission measurement takes longer than about 20 msec, and the mammal is agitated, precautions to insure immobilization of the mammal during photon emission measurement, commensurate with the degree of agitation of the mammal, need to be considered to preserve the spatial information in the constructed image. For example, in a case where the mammal is a person and photon emission measurement time is on the order of a few seconds, the mammal may simply be asked to remain as still as possible during photon emission measurement (imaging). On the other hand, if the mammal is an rodent, such as a mouse, the subject is immobilized using, for example, an anesthetic or a mechanical restraining device.

In cases where it is desired to measure only the total amount of light emanating from a mammal, the mammal does not necessarily need to be immobilized, even for long periods of photon emission measurements. All that is required is that the mammal be confined to the detection field of the photodetector during imaging. It will be appreciated, however, that immobilizing the mammal during such measuring may improve the consistency of results obtained, because the thickness of tissue through which detected photons pass will be more uniform from animal to animal.

Further Considerations During Imaging

The visualization of fluorescent light-generating proteins requires an excitation light source, as well as a photodetector. Furthermore, it will be understood that the excitation light source is turned on during the measuring of photon emission from the light-generating protein.

Appropriate selection of a fluorophor, placement of the light source and selection and placement of filters, all of which facilitate the construction of an informative image, are discussed above, in the section on fluorescent light-generating proteins.

High-Resolution Imaging.

Photon scattering by tissue limits the resolution that is obtained by imaging bioluminescent proteins through a measurement of total photon emission. It will be understood that the present invention also includes embodiments in which the light-generation of bioluminescent proteins is synchronized to an external source which is focused at selected points within the mammal, but which does not scatter significantly in tissue, allowing the construction of higher-resolution images. For example, a focused ultrasound signal is used to scan, in three dimensions, the mammal being imaged. Light-generation from areas which are in the focal point of the ultrasound is resolved from other photon emission by a characteristic oscillation imparted to the light by the ultrasound.

Constructing an Image of Photon Emission.

In cases where, due to an exceptionally bright bioluminescent protein and/or localization of light-generating fusion proteins near the surface of the mammal, a pair of “night-vision” goggles or a high sensitivity video camera was used to obtain an image, the image is simply viewed or displayed on a video monitor. If desired, the signal from a video camera is diverted through an image processor, which can store individual video frames in memory for analysis or printing, and/or can digitize the images for analysis and printing on a computer.

Alternatively, if a photon counting approach is used, the measurement of photon emission generates an array of numbers, representing the number of photons detected at each pixel location, in the image processor. These numbers are used to generate an image, typically by normalizing the photon counts (either to a fixed, pre-selected value, or to the maximum number detected in any pixel) and converting the normalized number to a brightness (greyscale) or to a color (pseudocolor) that is displayed on a monitor. In a pseudocolor representation, typical color assignments are as follows. Pixels with zero photon counts are assigned black, low counts blue, and increasing counts colors of increasing wavelength, on up to red for the highest photon count values. The location of colors on the monitor represents the distribution of photon emission, and, accordingly, the location of light-generating fusion proteins.

In order to provide a frame of reference for the conjugates, a greyscale image of the (still immobilized) mammal from which photon emission was measured is typically constructed. Such an image is constructed, for example, by opening a door to the imaging chamber, or box, in dim room light, and measuring reflected photons (typically for a fraction of the time it takes to measure photon emission). The greyscale image is constructed either before measuring photon emission, or after. The image of photon emission is typically superimposed on the greyscale image to produce a composite image of photon emission in relation to the mammal.

If it is desired to follow the localization and/or the signal from a light-generating conjugate over time, for example, to record the effects of a treatment on the expression, distribution and/or localization of a selected light-generating protein, the measurement of photon emission, or imaging is repeated at selected time intervals to construct a series of images. The intervals are as short as minutes, or as long as days or weeks.

Analysis of Photon Emission Images

Images generated by methods and/or using compositions of the present invention are analyzed by a variety of methods. They range from a simple visual examination, mental evaluation and/or printing of a hardcopy, to sophisticated digital image analysis. Interpretation of the information obtained from an analysis depends on the phenomenon under observation and the entity being used.

Models for the Study of Modulators of Cell Cycle, Cell Proliferation, and Cancer

The mammal- and cell-based systems described herein are utilized as models for the study of diseases, such as cancer. Animals of any species, including, but not limited to, mice, rats, rabbits, guinea pigs, pigs, micro-pigs, goats, and non-human primates, e.g., baboons, monkeys, and chimpanzees are used to generate disease animal models. In addition, cells from humans are used. These systems is used in a variety of applications. Such assays is utilized as part of screening strategies designed to identify compounds, such as compounds that are capable of ameliorating disease symptoms. Thus, the animal- and cell-based models are used to identify drugs, pharmaceuticals, therapies and interventions that are effective in treating disease.

Animal-based disease systems, such as those described herein, are used to identify compounds capable of ameliorating disease symptoms. Such animal models are used as test substrates for the identification of drugs, pharmaceuticals, therapies, and interventions that are effective in treating a disease or other phenotypic characteristic of the animal. For example, animal models are exposed to a compound or agent suspected of exhibiting an ability to ameliorate disease symptoms, at a sufficient concentration and for a time sufficient to elicit such an amelioration of disease symptoms in the exposed animals. The response of the animals to the exposure is monitored by assessing the reversal of disorders associated with the disease. Neonatal, juvenile, and adult animals are exposed.

More particularly, using the animal models of the invention, methods of identifying compounds are provided, in which such compounds are identified on the basis of their ability to affect an aspect of the cell cycle or cell proliferation involving E2F or an E2F responsive promoter.

Cell-based systems are used to identify compounds that may act to ameliorate disease symptoms. The cell or population of cells are isolated from one or more transgenic animals. Alternatively, the cells are recombinant cells produced by introducing a transgene (e.g. by infection, transfection, or other means). Cells are isolated from tumors or a tumor cell-containing tissue, organ, or individual. Alternatively, the cell or population of cells are contacted with agents (carcinogenic agents) that increase the tumorigenicity of the cells. For example, such cell systems are exposed to a compound suspected of exhibiting an ability to ameliorate disease symptoms, at a sufficient concentration and for a time sufficient to elicit such an amelioration of disease symptoms in the exposed cells. After exposure, the cells are examined to determine whether one or more of the disease cellular phenotypes has been altered to resemble a more normal or more wild-type, non-disease phenotype.

The present invention is employed in a process for screening for test compounds such as inducers, i.e., compounds that bind directly or indirectly to and modulate (e.g., either activate or repress) an E2F responsive promoter, or repressors, i.e., compounds that inhibit gene expression controlled by an E2F responsive promoter. In some embodiments, the compounds bind to polypeptides, such as transcription factions, that bind to an E2F responsive promotor. Alternatively, the compounds bind to polypeptides, such as enzymes, that modify DNA-binding proteins (such modifications include phosphorylation, dephosphorylation, proteolysis, acetylation, oxidation, carbonylation, cross-linking, and glycation). Thus, the compositions of the invention may also be used to assess the binding of putative transcription factors, small molecule mimetics, and other DNA-binding compounds in, for example, cells, cell-free preparations, chemical libraries, and natural product mixtures as known in the art. Any methods routinely used to identify and screen for compounds that can activate or repress E2F responsive promoters are used in accordance with the present invention.

The test compounds of the invention are obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. In embodiments of the invention, the library is a cDNA library, a peptide library, or an antibody library. See, e.g., Lam, 1997. Anticancer Drug Design 12:145.

Libraries of chemical and/or biological mixtures, such as fungal, bacterial, or algal extracts, are known in the art and are screened with any of the assays of the invention. Examples of methods for the synthesis of molecular libraries are found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds are presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

Pharmaceutical Compositions, Effective Dosages, and Routes of Administration

The identified compounds that modulate E2F responsive promoters are administered to a patient at therapeutically effective doses to treat or ameliorate a disease, such as cancer. A therapeutically effective dose refers to that amount of the compound sufficient to result in amelioration of symptoms of the disease.

Toxicity and therapeutic efficacy of such compounds are determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it is expressed as the ratio LD50/ED50. Compounds that exhibit large therapeutic indices are preferred. While compounds that exhibit toxic side effects are used, care should be taken to design a delivery system that targets such compounds to the site of affected tissue in order to minimize potential damage to uninfected cells and, thereby, reduce side effects.

The data obtained from the cell culture assays and animal studies are used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose is estimated initially from cell culture assays. A dose is formulated in animal models to achieve a circulating plasma concentration range that includes the IC50 (i.e., the concentration of the test compound that achieves a half-maximal inhibition of symptoms) as determined in cell culture. Such information is used to more accurately determine useful doses in humans. Levels in plasma are measured, for example, by high performance liquid chromatography.

Pharmaceutical compositions for use in accordance with the present invention are formulated in conventional manner using one or more physiologically acceptable carriers or excipients. Thus, the compounds and their physiologically acceptable salts and solvates are formulated for administration by inhalation or insufflation (either through the mouth or the nose) or oral, buccal, parenteral, topical, subcutaneous, intraperitoneal, intraveneous, intrapleural, intraoccular, intraarterial, or rectal administration. It is also contemplated that pharmaceutical compositions are administered with other products that potentiate the activity of the compound and optionally, may include other therapeutic ingredients.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets are coated by methods well known in the art. Liquid preparations for oral administration may take the form of, for example, solutions, syrups or suspensions, or they are presented as a dry product for constitution with water or other suitable vehicle before use. Such liquid preparations are prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration are suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit is determined by providing a valve to deliver a metered amount. Capsules and cartridges of e.g. gelatin for use in an inhaler or insufflator are formulated containing a powder mix of the compound and a suitable powder base such as lactose or starch.

The compounds are formulated for parenteral administration by injection, e.g., by bolus injection or continuous infusion. Formulations for injection are presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient is in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. Oral ingestion is possibly the easiest method of taking any medication. Such a route of administration, is generally simple and straightforward and is frequently the least inconvenient or unpleasant route of administration from the patient's point of view. However, this involves passing the material through the stomach, which is a hostile environment for many materials, including proteins and other biologically active compositions. As the acidic, hydrolytic and proteolytic environment of the stomach has evolved efficiently to digest proteinaceous materials into amino acids and oligopeptides for subsequent anabolism, it is hardly surprising that very little or any of a wide variety of biologically active proteinaceous material, if simply taken orally, would survive its passage through the stomach to be taken up by the body in the small intestine. The result is that many proteinaceous medicaments must be taken in through another method, such as parenterally, often by subcutaneous, intramuscular or intravenous injection.

Pharmaceutical compositions may also include various buffers (e.g., Tris, acetate, phosphate), solubilizers (e.g., Tween, Polysorbate), carriers such as human serum albumin, preservatives (thimerosol, benzyl alcohol) and anti-oxidants such as ascorbic acid in order to stabilize pharmaceutical activity. The stabilizing agent is a detergent, such as tween-20, tween-80, NP-40 or Triton X-100. EBP may also be incorporated into particulate preparations of polymeric compounds for controlled delivery to a patient over an extended period of time. A more extensive survey of components in pharmaceutical compositions is found in Remington's Pharmaceutical Sciences, 18th ed., A. R Gennaro, ed., Mack Publishing, Easton, Pa. (1990).

In addition to the formulations described previously, the compounds may also be formulated as a depot preparation. Such long acting formulations are administered by implantation (for example, subcutaneously or intramuscularly) or by intramuscular injection. Thus, for example, the compounds are formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device is accompanied by instructions for administration.

The following examples are intended only to illustrate the present invention and should in no way be construed as limiting the subject invention.

### EXAMPLES

### Example 1

### Generation of Transgenic Mice Expressing a Nucleic Acid Encoding a Bioluminescent Protein Under the Control of an E2F Responsive Promoter by Homologous Recombination

Transgenic mice containing an E2F responsive promoter and a nucleic acid encoding firefly luciferase (E2F-Luc) are generated and used to detect proliferating cells. A nucleic acid containing an E2F responsive promoter upstream of a nucleic acid encoding luciferase is cloned into the pBig T vector, thereby placing it downstream of a floxed neo tPA (triple polyadenylation site) cassette. A restriction fragment spanning the Neo cassette and the luciferase transcriptional unit is then be subcloned into pROSA26PA, which contains flanking sequences from the ROSA 26 locus, to facilitate homologous recombination. The targeting plasmid is introduced (e.g., by electroporation) into embryonic stem cells (ES cells). Drug-resistant colonies (e.g., G418- and diphteria toxin-resistant) are selected and screened for homologous recombination. Selected ES clones are injected into C57/BL6 blastocysts, and implanted into pseudopregnant mothers. Chimeric mice are genotyped by Southern blot assay and bred to wild-type C57/BL6 mice to ensure germ line transmission. The E2F-Luc mice are crossed to transgenic mice expressing Cre from the E2A promoter to eliminate the Neo/tPA cassette. Cre(+); E2F-Luc mice are backcrossed to C57/BL6 mice to eliminate the Cre gene. E2F-Luc mice are treated with luciferin and drugs that directly or indirectly modulate E2F activities.

### Example 2

### Generation of Transgenic Mice Expressing a Nucleic Acid Encoding a Bioluminescent Protein Under the Control of an E2F Responsive Promoter by Pronuclear Injection

A DNA plasmid vector E2F-Luc containing the human E2F1 promoter, a cDNA sequence encoding firefly luciferase, and a polyadenylation signal on the 3′ end are generated. Transgenic mice are generated by pronuclear microinjection using fertilized eggs of the FVB/N strain (Taconic). See, e.g., U.S. Pat. No. 4,873,191 and “Manipulating the Mouse Embryo; A Laboratory Manual” 2nd edition (eds., Hogan, Beddington, Costantimi and Long, Cold Spring Harbor Laboratory Press, 1994; each of which is incorporated herein by reference in its entirety). Founder mice are identified by PCR analysis of DNA prepared from tail biopsies collected at weaning, using luciferase-specific primers. Northern Blot analysis is performed to confirm expression of the transgenic luciferase mRNA. Total RNA is isolated from whole brain of 3-month-old transgenic and nontransgenic mice according to the method of Chomczynski et al., Anal. Biochem. 162, 156 (1987) or by other standard methods.

### Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents are selected for the present invention and embodiments thereof.

What is claimed is:

1. A transgenic mammal comprising a recombinant nucleic acid molecule stably integrated into the genome of said mammal, said recombinant nucleic acid molecule comprising an E2F responsive promoter operably linked to a nucleic acid encoding a bioluminescent protein.

2. The mammal of claim 1, wherein said E2F responsive promoter is capable of binding a polypeptide selected from the group consisting of pRB, p107, p130, E2F1, E2F2, E2F3, E2F4, E2F5, E2F6, E2F7, G1 cyclin, and a cyclin-dependent kinase.

3. The mammal of claim 1, wherein said E2F responsive promoter is selected from the group of promoters consisting of E2F-1 promoter, dihydrofolate reductase promoter, DNA polymerase alpha promoter, c-myc promoter, and B-myb promoter.

4. The mammal of claim 1, wherein said E2F responsive promoter is human E2F-1 promoter.

5. The mammal of claim 1, wherein said E2F responsive promoter comprises SEQ ID NO: 1.

6. The mammal of claim 1, wherein said bioluminescent protein is selected from the group consisting of ferredoxin IV, green fluorescent protein, red fluorescent protein, yellow fluorescent protein, the luciferase family, and the aequorin family.

7. The mammal of claim 1, wherein said recombinant nucleic acid molecule is of human or murine origin.

8. An isolated cell of the mammal of claim 1.

9. The cell of claim 8, wherein said cell is a stem cell, a germ cell, a precursor cell or a progenitor cell.

10. The mammal of claim 1, wherein said mammal is a mouse.

11. The mammal of claim 2, wherein binding of said polypeptide to said E2F responsive promoter increases the production of said bioluminescent protein.

12. The mammal of claim 1, wherein said mammal is a mouse, and said E2F responsive promoter comprises SEQ ID NO: 1 and said bioluminescent protein is luciferase.

13. A method for the production of a transgenic mammal, comprising introduction of a recombinant nucleic acid molecule into a germ cell, an embryonic cell, an egg cell or a cell derived therefrom, said recombinant nucleic acid molecule comprising an E2F responsive promoter operably linked to a nucleic acid encoding a bioluminescent protein.

14. The method of claim 13, wherein said mammal is a rodent.

15. The method of claim 14, wherein said mammal is a mouse.

16. A method for the identification of a compound capable of modifying an activity of E2F, comprising:
a) contacting the transgenic mammal of claim 1 or a cell isolated therefrom with a test compound; and
b) measuring the effect of said test compound on said E2F responsive promoter, thereby identifying a compound that modifies an activity of E2F.

17. The method of claim 16, wherein said compound increases E2F binding to an E2F responsive promoter.

18. The method of claim 16, wherein said compound decreases E2F binding to an E2F responsive promoter.

19. A method for detecting a proliferating cell, comprising:
a) admininistering luciferin to the mouse of claim 12; and
b) measuring with a photodetector device, photon emission through opaque tissue, thereby detecting a proliferating cell.

20. A method for detecting a proliferating cell, comprising:
a) contacting an isolated nucleic acid and a cell suspected of being a proliferating cell, said nucleic acid comprising an E2F responsive promoter operably linked to a nucleic acid encoding a bioluminescent protein, such that the nucleic acid enters into said cell and said bioluminescent protein is produced; and
b) detecting said produced bioluminescent protein,
thereby detecting the proliferating cell.

21. The method of claim 20, wherein the proliferating cell is a cancer-associated cell.

22. The method of claim 21, wherein the cancer is selected from the group consisting of brain cancer, glioma, lung cancer, liver cancer, breast cancer, Burkitts lymphoma, Hodgkin's disease, cervical cancer, ovarian cancer, and retinoblastoma.

23. The method of claim 20, wherein the nucleic acid further comprises a vector.

24. The method of claim 23, wherein said vector is selected from the group consisting of plasmid vectors and viral vectors.

25. The method of claim 20, wherein said E2F responsive promoter is capable of binding a polypeptide selected from the group consisting of pRB, p107, E2F1, E2F2, E2F3, E2F4, E2F5, E2F6, G1 cyclin, a cyclin-dependent kinase and E1A.

26. The method of claim 20, wherein said E2F responsive promoter is selected from the group of promoters consisting of E2F-1 promoter, dihydrofolate reductase promoter, DNA polymerase alpha promoter, c-myc promoter, and B-myb promoter.

27. The method of claim 20, wherein said E2F promoter comprises SEQ ID NO: 1.

28. The method of claim 20, wherein said bioluminescent protein is selected from the group consisting of ferredoxin IV, green fluorescent protein, red fluorescent protein, yellow fluorescent protein, the luciferase family, and the aequorin family.

29. A non-invasive method for localizing a malignant cell in a subject, comprising:
a) introducing to the subject a nucleic acid comprising an E2F responsive promoter operably linked to a nucleic acid encoding a bioluminescent protein, such that the nucleic acid enters into said cell and said bioluminescent protein is produced; and
b) detecting the bioluminescent protein,
thereby localizing said malignant cell in said subject.

30. A method for localizing cancerous tissue in a subject, comprising:
a) introducing to the subject a nucleic acid comprising an E2F responsive promoter operably linked to a nucleic acid encoding a bioluminescent protein, such that the nucleic acid enters into one or more cells of the cancerous tissue, and said bioluminescent protein is produced in said one or more cells; and
b) detecting the bioluminescent protein,
thereby localizing said cancerous tissue in said subject.

31. A method of determining the efficacy of an anti-tumor compound in a subject, comprising:
a) introducing to the subject a nucleic acid comprising an E2F responsive promoter operably linked to a nucleic acid encoding a bioluminescent protein, such that the nucleic acid enters into one or more cells of the tumor tissue and said bioluminescent protein is produced in said one or more cells;
b) measuring the luminescence of said bioluminescent protein prior to administration of the anti-tumor compound;
c) administering the anti-tumor compound to the subject;
d) measuring the luminescence of said bioluminescent protein following administration of the anti-tumor compound; and
e) comparing the luminescence of said bioluminescent protein prior to and following administration of the anti-tumor compound,
thereby determining the efficacy of said anti-tumor compound in said subject.

32. A method for the identification of a compound capable of modifying an activity of E2F, comprising:
a) contacting an isolated cell containing a nucleic acid comprising an E2F responsive promoter operably linked to a nucleic acid encoding a bioluminescent protein with a test compound; and
b) measuring the effect of said test compound on said E2F responsive promoter, thereby identifying a compound that modifies an activity of E2F.

33. The method of claim 32, wherein said compound increases E2F binding to an E2F responsive promoter.

34. The method of claim 32, wherein said compound decreases E2F binding to an E2F responsive promoter.
